(19) United States
(12) Patent Application Publication
(10) Pub. No.: US 2018/0028568 A1
(43) Pub. Date: Feb. 1, 2018

(54) CELL-BASED COMPOSITION AND USE THEREOF FOR TREATMENT OF PSORIASIS AND AUTOIMMUNE DISEASES

(71) Applicant: Cytopeutics Sdn. Bhd., Selangor (MY)

(72) Inventors: Bernard Sze Piaw Chin, Selangor (MY); Kong Yong Then, Selangor (MY); Soon Keng Cheong, Selangor (MY)

(21) Appl. No.: 15/599,052

(22) Filed: May 18, 2017

(30) Foreign Application Priority Data
Jul. 26, 2016 (MY) PI 2016702711

(51) Int. Cl. A61K 35/28; A61K 9/00

(52) U.S. Cl. CPC A61K 35/28; A61K 9/0019

(57) ABSTRACT

A method for the treatment of psoriasis or an autoimmune disease in a subject by administering to said subject an effective amount of a cell-based composition containing a suspension of mesenchymal stem cells in crystalloid with a cellular concentration from 0.01 million to 3.0 million cells/ml.

### CROSS-REFERENCE TO RELATED APPLICATIONS

The present application claims the benefit of Malaysian Application No. PI 2016702711, filed on Jul. 26, 2016, the contents of which are hereby incorporated by reference in their entirety.

### FIELD OF INVENTION

The present invention relates to a cell-based composition and use thereof for the treatment of psoriasis and autoimmune diseases including systemic lupus erythematosus.

### BACKGROUND OF THE INVENTION

Psoriasis is an autoimmune and systemic inflammatory disease which affects primarily skin, nails and occasionally joints. Patients afflicted with psoriasis are typically seen with appearance of red, flaky, crusty patches of skin covered with silvery scales on their bodies. Severity of psoriasis varies significantly from one to another. In chronic cases, psoriasis can have a massive impact on quality of life of the patient.

Various treatment methods have been developed in the past for treatment of psoriasis. Conventionally, the common ways of managing psoriasis which are standard systemic therapies include phototherapy, steroids and topical treatment amongst others. However, these methods may not necessarily confer overall health benefits to the patient nor reduce possibility of recurrence of the disease.

In light of the above, alternative ways for treating psoriasis which are safer and clinically effective became focus of investigation for medical experts. Studies on mesenchymal stem cells, also known as mesenchymal stromal cells, have generated a lot of interest among researchers and clinicians due to its attributes which include ability to reduce inflammation and regulate immune response.

However, studies and research publications on treating psoriasis using cell-based methods in humans appears to be lacking. Accordingly, there remains a need for a novel cell-based composition which is clinically safe and therapeutically effective for the treatment of psoriasis and autoimmune diseases in humans.

### SUMMARY OF THE INVENTION

In overcoming limitations resulting from conventional methods in the past, the present invention provides a cell-based preparation and its use thereof which is clinically safe and therapeutically effective for psoriasis and autoimmune diseases.

More particularly, the present invention relates to a cell-based composition comprising a suspension of mesenchymal stem cells in crystalloid with a cellular concentration from 0.01 million to 3.0 million cells/ml wherein the cell-based composition is used in a form of medicament for treatment of psoriasis and autoimmune diseases including systemic lupus erythematosus.

Thus, one aspect of this invention is a method for treating psoriasis or autoimmune diseases (e.g., systemic lupus erythematosus) in a subject by administering to the subject (e.g., by intradermal or intravenous injection) the cell-based composition described above and hereinafter in a therapeutically effective amount (e.g., 0.25 million to 3.0 million cells/kg body weight).

### BRIEF DESCRIPTION OF DRAWINGS

The drawings constitute part of this specification and include an exemplary or preferred embodiment of the invention, which may be embodied in various forms. It should be understood, however, that the disclosed preferred embodiments are merely exemplary of the invention. Therefore the figures disclosed herein are not to be interpreted as limiting, but merely as the basis for teaching one skilled in the art of the invention.

In the appended drawings:

FIG. 1 illustrates morphology of human umbilical cord-derived mesenchymal stem cells.

FIG. 2 illustrates immunophenotyping assay results of the mesenchymal stem cells.

FIG. 3 illustrates adipogenesis, osteogenesis and chondrogenesis of the mesenchymal stem cells respectively.

FIG. 4 illustrates results before and after one infusion and intradermal injections of cell-based composition.

FIG. 5 illustrates results at baseline and 6 months after one infusion and intradermal injections of cell-based composition.

FIG. 6 illustrates regular infusion of cell-based composition induces long-term remission in patients with autoimmune psoriasis.

FIG. 7 illustrates results before and after multiple injections of cell-based composition.

### DETAILED DESCRIPTION OF THE INVENTION

A detailed description of the present invention is described herein. The present invention is directed to a cell-based composition and use thereof for treating psoriasis and autoimmune diseases including systemic lupus erythematosus. More particularly, the present invention relates to a cell-based composition comprising a suspension of mesenchymal stem cells in crystalloid with a cellular concentration from 0.01 million to 3.0 million cells/ml wherein the cell-based composition is used in a form of medicament for treatment of psoriasis and autoimmune diseases including systemic lupus erythematosus.

Accordingly, the mesenchymal stem cells used for preparation of the suspension are derived from various sources including, but not limiting to, human umbilical cord, bone marrow, fat tissue, peripheral blood or tooth pulp. Samples are either collected from mothers post-birth or from healthy donors. If required, the samples are cleaned and disinfected accordingly.

Upon collection, the samples are sent to laboratory to be processed further. The samples will be digested using a digestion enzyme, preferably, but not limiting to collagenase type II and followed by centrifugation, leaving a layer of supernatant and pellet containing mesenchymal stem cells. The mesenchymal stem cells are isolated and cultured in a specially formulated medium supplemented with a combination of various antibiotics and animal-free serum. The cultures are maintained at a temperature range from 35° C. to 40° C., preferably at 37° C. in a humidified atmosphere for 3-4 days.

As it will be apparent to a person of ordinary skill in the art, mesenchymal stem cells are adherent to plastic. Non-adherent cells are discarded and the growth medium is replaced every 3-4 days until the cells reached confluence.

Upon reaching 70%-80% confluence, the adherent mesenchymal stem cells are incubated with a dissociation enzyme, preferably, but not limiting to trypsin and re-plated at 1×10⁴ cells/ml for a series of passages, preferably, but not limiting to 3-4 passages. The mesenchymal stem cells are then harvested in a culture flasks, thus expanding population of the cells.

The mesenchymal stem cells are characterized in accordance to a criteria set forth by International Society for Cellular Therapy (ISCT). Apart from adherence to plastic, established criteria defining mesenchymal stem cells include expression of antigen markers as measured by flow cytometry and tri-differentiation ability of the cells (Dominici 2006). Using flow cytometry, the mesenchymal stem cells are defined by expression of CD73, CD90, and CD105 markers whilst absence of expression for CD34, CD45, and HLA-DR markers. Meanwhile, the tri-differentiation ability of the mesenchymal stem cells is demonstrated by way of the cells differentiating into osteoblasts, adipocytes and chondroblasts.

Once the population of mesenchymal stem cells have been expanded, a disassociation enzyme, preferably, but not limiting to trypsin is added in to the flasks and incubated at a temperature range from 35° C. to 40° C., more preferably at 37° C., for a period of 1-15 minutes, more preferably for 5 minutes to detach the plastic-adherent mesenchymal stem cells, leaving the cells slightly shrunk. Next, the flasks are gently tapped to dislodge the cells and medium is further added to dilute the trypsin, forming a mesenchymal cell suspension. The cell suspension is then transferred into 50 ml centrifuge tubes and centrifuged at a speed range from 300 g to 800 g, more preferably at 500 g at a temperature range from 18° C. to 20° C. for 10 minutes forming a layer of supernatant with pellet at bottom of the tubes. The supernatant is removed, leaving the pellet of mesenchymal stem cells in the tubes.

The pellet of mesenchymal stem cells are re-suspended in a sterile cryovial of 1.8 ml in size containing cryopreservation medium comprising from 80% to 90% animal-free serum and a cryoprotectant, preferably, but not limiting to dimethyl sulfoxide from 1% to 10%. Alternatively, dimethyl sulfoxide may also be substituted with human serum albumin. Typically, a cryovial contains from 25 million to 30 million cells per vial. Alternatively, cryovials of up to 5 ml in size may also be used.

The mesenchymal stem cells in cryovials are frozen in a controlled rate freezer until −70 to −90° C. but preferably −90 gradually before transferring into quarantine tank, preferably, but not limiting to a vapour phase liquid nitrogen storage tank.

To prepare a cell-based composition for the treatment of psoriasis and autoimmune diseases including systemic lupus erythematosus, the cryopreserved mesenchymal stem cells in cryovials are first thawed at a temperature ranging from 30° C. to 40° C., preferably at 37° C. in a water bath or an incubator for a period from 1 to 5 minutes, more preferably at 2 minutes. Next, the cells are then transferred into new sterile cryovials and are washed with sterile saline, preferably but not limiting to 0.9% sodium chloride. The washed cells in the sterile cryovials will then be centrifuged at a speed ranging from 500×g to 1000×g, preferably at 800×g for a period from 3 to 10 minutes, more preferably for 5 minutes at room temperature, forming a layer of supernatant and a pellet of mesenchymal stem cells.

Typically, the supernatant is removed and discarded, leaving the pellet of mesenchymal stem cells in the cryovial. The pellet of mesenchymal stem cells are then re-suspended with sterile saline at a volume ranging from 5 to 20 ml, preferably at 10 ml, forming a suspension of mesenchymal stem cells.

A cell-based composition is then prepared by infusing the suspension of mesenchymal stem cells into crystalloid to reach a cellular concentration from 0.01 million to 3.0 million cells/ml. The crystalloid includes, but not limiting to normal or half-normal saline or colloid.

Exact amount of cells per kg body weight to be administered into a patient depends on variety of factors including body weight, route of administration, age and gender of the patient, and also the type of mechanism of action targeted. Typically, the therapeutically effective amount of the cell-based composition used for the treatment of psoriasis and autoimmune diseases including systemic lupus erythematosus is from to 0.25 million to 3.0 million cells/kg body weight.

The following examples further illustrate but by no means limit the scope of the invention:

### Example 1: Collection and Handling of Umbilical Cord Sample

The umbilical cord sample was detached from placenta of a donor post-birth using medical scissors and was immediately submerged in povidone iodine solution for 1-5 minutes to eliminate bacteria and to avoid any risk of contamination. Alternatively, the umbilical may be disinfected using alcohol swab. Upon disinfection, the umbilical cord was then placed in a sterile container of sterile saline solution to maintain moisture. Subsequently, the sterile container was placed into a collection kit and was transported to laboratory using a thermo-insulated bag and kept under a temperature range from 4° C. to 37° C.

The sample was then processed within 48 hours from time of collection.

### Example 2: Isolation and Culture of Mesenchymal Stem Cells

First, veins and arteries of the umbilical cord were removed and followed by mincing into 1-2 mm fragments. The fragments were digested with an enzyme, preferably, but not limiting to 0.01% to 0.05% collagenase type II, for a period from 1 to 3 hours, forming a mixture. Next, a centrifugation was carried out to separate the mesenchymal stem cells from the mixture. The mesenchymal stem cells were isolated and then cultured in a growth medium, preferably, but not limiting to Dulbecco's Modified Eagle's Medium (DMEM) which may or may not contain low glucose supplemented with 5-20% animal-free serum and a combination of antibiotics comprising 100 U/mL penicillin, 100 mg/mL streptomycin, 250 ng/mL amphotericin B and 2 mM glutamine. The cultures were maintained at 37° C. in a humidified atmosphere of 5% CO₂ and 95% air for 3 days. Non-adherent cells were discarded and the growth medium was replaced every 3-4 days until the cells reached confluence.

Next, the plastic-adherent mesenchymal stem cells were incubated with trypsin and re-plated at 1×10⁴ cells/ml for 3-4 passages. The mesenchymal stem cells were then harvested in a culture flasks, thus expanding population of the cells.

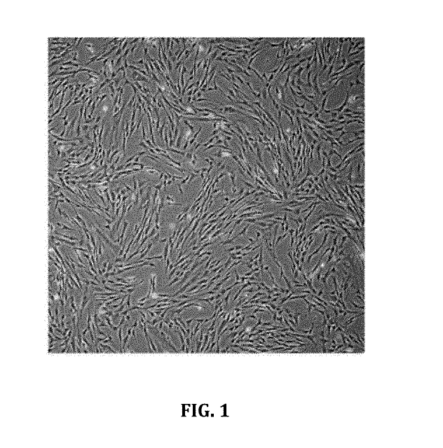

FIG. 1 illustrates the morphology of the mesenchymal stem cells.

### Example 3: Characterization of Mesenchymal Stem Cells

Immunophenotyping

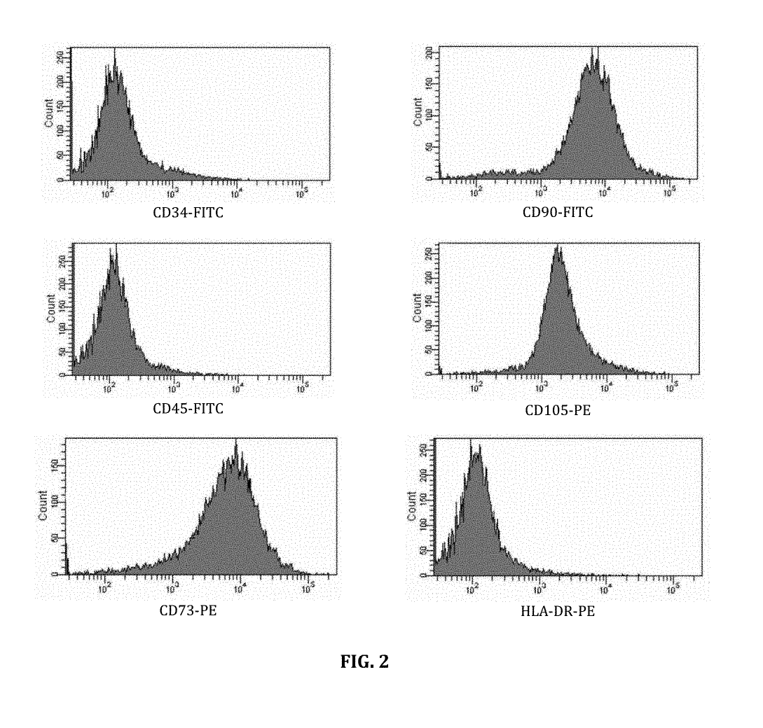

The mesenchymal stem cells were immunophenotyped at passage three using isotype (fluorescein isothiocyanate) FITC and (phycoerythrin) PE controls with antigen markers which include CD34, CD45, CD73, CD90, CD105 and HLA-DR. As shown in FIG. 2, the immunophenotyping assay results for the mesenchymal stem cells validate expression for CD73, CD90 and CD105 whilst lacking expression for CD34, CD45 and HLA-DR.

Differentiation Assay

To perform this assay, a selection of specially formulated differentiation medium were used to induce tri-differentiation ability of the mesenchymal stem cells.

Adipogenesis:

The mesenchymal stem cells were treated in adipogenic differentiation medium comprising complete medium supplemented with 1 mM dexamethasone and 0.2 mM indomethacin, 0.01 mg/mL insulin and 0.5 mM 3-isobutil-1-metil-xantina. The medium was replaced every 3 days, and the differentiated cells were subjected to Oil Red O staining after about 14 days of culture.

Chondrogenesis:

The mesenchymal stem cells were cultured in pellet form and maintained in a chemically defined basal medium comprising complete medium supplemented with 50 mg/mL ascorbate-2-phosphate, 1.0 mM sodium piruvate, 40 mg/mL proline, 10 ng/mL transforming growth factor-b3, 6.25 mg/mL human insulin, 6.25 mg/mL transferrin, 6.25 mg/mL bovine insulin, 6.25 mg/mL selenous acid, 1.25 mg/mL linoleic acid, and 5.35 mg/mL bovine serum albumin. Next, the cells were suspended in 1 mL of chondrogenic medium and replaced every 3-4 days. Chondrogenic pellets were harvested after 5 weeks in culture. To assess chondrogenesis, Alcian Blue was used to stain cartilage matrix.

Osteogenesis:

The mesenchymal stem cells were treated in osteogenic differentiation medium comprising complete medium supplemented with 50 mg/mL ascorbate-2-phosphate, 10 mM b-glycerophosphate, and 100 nM dexamethasone. The medium was replaced every 3 days continuously for 2-3 weeks. Alizarin Red S was used to stain matrix mineralization associated with differentiated osteocytes.

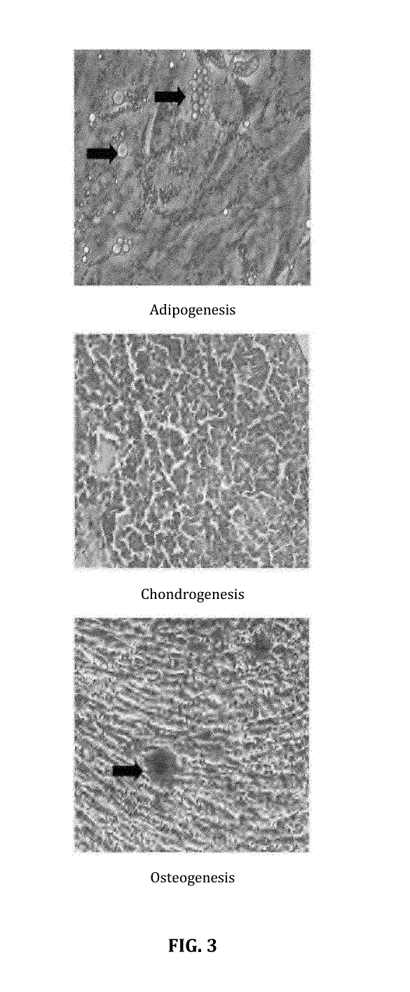

FIG. 3 demonstrates the tri-differentiation ability of the mesenchymal stem cells exhibiting adipogenesis, osteogenesis and chondrogenesis respectively.

### Example 4: Cryopreservation of Mesenchymal Stem Cells

Once the population of mesenchymal stem cells was expanded, trypsin was added in to the flasks and incubated at 37° C., for 5 minutes to detach the plastic-adherent mesenchymal stem cells, leaving the cells slightly shrunk. Next, the flasks were gently tapped to dislodge the cells and medium was further added to dilute the trypsin, forming a mesenchymal cell suspension. The cell suspension was then transferred into 50 ml centrifuge tubes and centrifuged at 500 g at a temperature ranging from 18° C. to 30° C. for 10 minutes forming a layer of supernatant with pellet at bottom of the tubes. The supernatant was removed, leaving the pellet of mesenchymal stem cells in the tubes.

The pellet of mesenchymal stem cells were re-suspended in a sterile cryovial containing cryopreservation medium comprising up to 90% animal-free serum and up to 10% dimethyl sulfoxide and were cryopreserved in a controlled freezing gradual rate at −90° C. before being transferred into a quarantine tank at −190° C.

### Example 5: Preparation of the Cell-Based Composition for Treatment

The cryopreserved mesenchymal stem cells in cryovials were thawed at 37° C. in a water bath or an incubator for 2 minutes. Next, the cells were then transferred into new sterile cryovials and were washed with 0.9% sodium chloride. The washed cells in the sterile cryovials were then centrifuged at 800×g for 3-10 minutes at room temperature, forming a layer of supernatant and a pellet of mesenchymal stem cells. The supernatant was removed using a sterile syringe, leaving the pellet of mesenchymal stem cells in the cryovial.

The pellet of mesenchymal stem cells was then re-suspended with sterile saline at 10 ml, forming a suspension of mesenchymal stem cells. A cell-based composition was then prepared by infusing the suspension of mesenchymal stem cells into saline at a volume of 250 ml, in a sterile bottle. The cells may also be suspended in much smaller volumes of 5-10 ml at a concentration of 1.0-10.0×10⁶ cells per ml and injected intradermally into the lesions.

### Example 6: Treatment Procedure Using the Cell-Based Composition

A pilot study was carried out on 5 patients with autoimmune skin conditions (4 psoriasis and 1 systemic lupus erythematosus). All patients are aged between 18-70 years old, with confirmed diagnosis of psoriasis or SLE and are refractory or intolerant to immunosuppresive medications such as prednisolone, methrotrexate, cyclosporine and sulphasalazine. All patients received intradermal direct lesion injection of the cell-based composition followed by intravenous administration of the cell-based composition. Patients' condition was compared by visual determination of size and distribution of lesions before and after treatment.

### Example 7: Results and Discussion

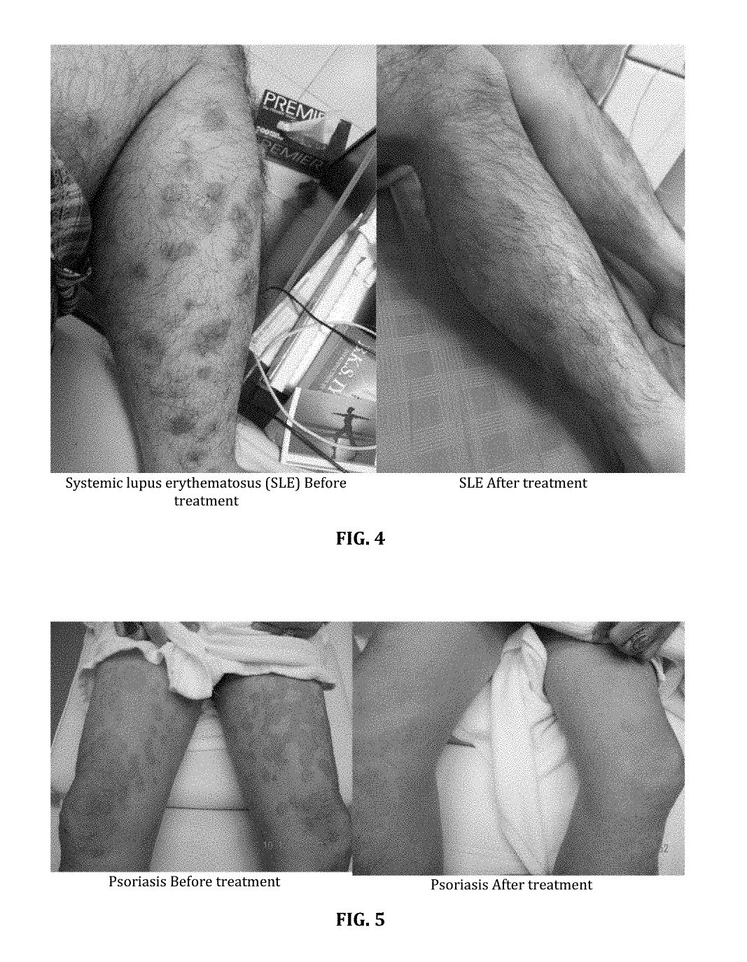

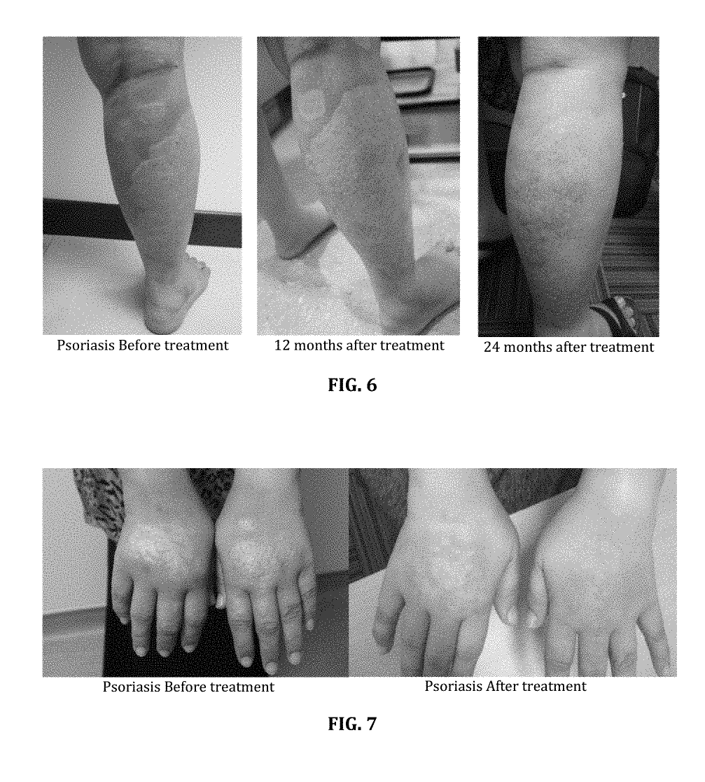

FIGS. 4-7 illustrate the resolution of lesions before and after treatment for psoriasis and systemic lupus erythematosus.

Having described preferred embodiments of the present invention with reference to the accompanying drawings, it is not intended that these embodiments and examples illustrate and describe all possible forms of the present invention, and it is to be understood that the present invention is not limited to those precise embodiments, and that various changes and modifications may be effected therein by one skilled in the art without departing from the scope of the invention as defined in the appended claims.

1. A method for the treatment of psoriasis or an autoimmune disease in a subject, said method comprising administering to said subject a therapeutically effective amount of a cell-based composition containing a suspension of mesenchymal stem cells in crystalloid with a cellular concentration from 0.01 million to 3.0 million cells/ml.

2. The method as claimed in claim 1, wherein the cell-based composition is administered by way of intradermal or intravenous injection.

3. The method as claimed in claim 1, wherein the therapeutically effective amount is from 0.25 million to 3.0 million cells/kg body weight.

4. The method as claimed in claim 1, wherein the suspension of mesenchymal stem cells is derived from cryopreserved mesenchymal stem cells containing from 25 million to 35 million cells per cryovial.

5. The method as claimed in claim 4, wherein the cell-based composition is administered by way of intradermal or intravenous injection.

6. The method as claimed in claim 5, wherein the therapeutically effective amount is from 0.25 million to 3.0 million cells/kg body weight.

7. The method as claimed in claim 1, wherein the suspension of mesenchymal stem cells is derived from human umbilical cord, bone marrow, fat tissue, peripheral blood or tooth pulp.

8. The method as claimed in claim 7, wherein the cell-based composition is administered by way of intradermal or intravenous injection.

9. The method as claimed in claim 8, wherein the therapeutically effective amount is from 0.25 million to 3.0 million cells/kg body weight.

10. The method as claimed in claim 1, wherein the suspension of mesenchymal stem cells is positive for a selected group of surface markers including CD73, CD90, and CD105, and negative for a selected group of surface markers including CD34, CD45, and HLA-DR.

11. The method as claimed in claim 10, wherein the cell-based composition is administered by way of intradermal or intravenous injection.

12. The method as claimed in claim 11, wherein the therapeutically effective amount is from 0.25 million to 3.0 million cells/kg body weight.

13. The method as claimed in claim 1, wherein the autoimmune disease is systemic lupus erythematosus.

14. The method as claimed in claim 2, wherein the autoimmune disease is systemic lupus erythematosus.

15. The method as claimed in claim 3, wherein the autoimmune disease is systemic lupus erythematosus.

16. The method as claimed in claim 4, wherein the autoimmune disease is systemic lupus erythematosus.

17. The method as claimed in claim 5, wherein the autoimmune disease is systemic lupus erythematosus.

18. The method as claimed in claim 6, wherein the autoimmune disease is systemic lupus erythematosus.

19. The method as claimed in claim 7, wherein the autoimmune disease is systemic lupus erythematosus.

20. The method as claimed in claim 8, wherein the autoimmune disease is systemic lupus erythematosus.

21. The method as claimed in claim 9, wherein the autoimmune disease is systemic lupus erythematosus.

22. The method as claimed in claim 10, wherein the autoimmune disease is systemic lupus erythematosus.

23. The method as claimed in claim 11, wherein the autoimmune disease is systemic lupus erythematosus.

24. The method as claimed in claim 12, wherein the autoimmune disease is systemic lupus erythematosus.
